(19) United States
(12) Patent Application Publication
(10) Pub. No.: US 2018/0292378 A1
(43) Pub. Date: Oct. 11, 2018

(54) DETECTION OF PEROXIDE BASED EXPLOSIVES

(71) Applicant: Steven J. Baker, Morrisville, NC (US)

(72) Inventor: Steven J. Baker, Morrisville, NC (US)

(21) Appl. No.: 15/484,684

(22) Filed: Apr. 11, 2017

(51) Int. Cl. G01N 33/22; G01N 33/00

(52) U.S. Cl. CPC G01N 33/227; G01N 33/0036

(57) ABSTRACT

The present invention is a system for testing for TATP residue on a person or object, without having to pull the person or object out of line, that can be done quickly, accurately, is non-invasive and does not require a physical contact.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

### Field of the Invention

The present invention relates to a quick detection system for the presence of an acetone peroxide residue, especially TATP (triacetone-triperoxide), its explosive relatives, and masking agents. More particularly, it relates to a system which quickly and accurately detects TATP present on individuals or objects.

### Description of Related Art

Acetone peroxide type explosives are easily obtained and manufactured, and thus have become an explosive of choice for use by terrorists. The use of such explosives has caused great concern at airports and other facilities located around the globe where terrorism is an issue. Security agencies have found it necessary to search passengers for its presence. The primary detection systems, in high volume passenger screening, as well as cargo screening, currently rely on metal detection, x-ray and physical examination. The screening for TATP is performed secondarily via physically swabbing individuals and articles to acquire a sample for analysis by gas chromatography. This practice delays each subject for a period of time.

Physically checking for TATP is normally much more invasive than primary screening, requiring pulling a passenger, luggage, or other object out of line, followed by swabbing (physical contact) in several places. The sample then has to be transferred to a gas chromatography device therefore, this part of the process is so time consuming, no matter how quick the analysis itself ends up being, the time frame for accomplishing screening means only a small percentage of passengers or objects are ever subjected to testing for TATP. A more remote system of detecting TATP was introduced in 2005. It requires use of multiple lasers shining on individuals to create a cloud of TATP, which can then be detected. The danger of using lasers on humans associated with this method, as well as poor collection rates, has rendered it not practically usable. It also does not avoid the problem of requiring to have a physical contact with the passenger. Accordingly, a system and method of quickly testing for the presence of TATP without any of the above problems is still of interest. Recently, the United States government has called on inventors to help with a detection system that can be utilized in a non-invasive high throughput screen, further emphasizing the need for new detection systems for TATP.

### BRIEF SUMMARY OF THE INVENTION

The present invention relates to a system which overcomes the problems described above. In particular, it has other attributes not found in prior testing systems, for example, the system is non-invasive, can be used with individual, groups of people or articles, such as luggage, quickly and timely, and can be integrated into other detection system enclosures. As such, it meets the current unmet needs demanded by airport screeners.

The present discovery that if one or more passengers or objects are placed in an enclosed area and the temperature is at least about 90 degrees F., enough TATP sublimates to gas for detection by withdrawing an air sample (in some cases concentrating the sample) and testing that air sample for sublimated TATP.

Accordingly, in one embodiment, there is a system for the detection of a triacetone-triperoxide residue on one or more subjects comprising:
a) an enclosed sampling chamber for positioning the subject;
b) a temperature regulator for providing a temperature of at least about 90 degrees F. in the sampling chamber;
c) an air sampler for taking air from the chamber after a dwell time of at least about 5 seconds; and
d) a system for concentrating the air sample and feeding it to a TATP detector system.

In yet another embodiment, there is a method for the detection of a triacetone-triperoxide residue on one or more subjects comprising:
a) providing an enclosed sampling chamber for positioning the subject;
b) providing a temperature regulator for providing a temperature of at least about 90 degrees F. in the sampling chamber;
c) providing an air sampler which takes an air sample from the chamber after a dwell time of at least about 5 seconds; and
d) concentrating the air sample and feeding it to a TATP detector system.

### BRIEF DESCRIPTION OF THE DRAWINGS

FIG. 1 is a diagram of the present system.

FIG. 2 is a flow chart of the method of the present invention.

### DETAILED DESCRIPTION OF THE INVENTION

While this invention is susceptible to embodiment in many different forms, there is shown in the drawings and will herein be described in detail specific embodiments, with the understanding that the present disclosure of such embodiments is to be considered as an example of the principles and not intended to limit the invention to the specific embodiments shown and described. In the description below, like reference numerals are used to describe the same, similar, or corresponding parts in the several views of the drawings. This detailed description defines the meaning of the terms used herein and specifically describes embodiments in order for those skilled in the art to practice the invention.

### Definitions

The terms “about” and “essentially” mean±10 percent.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

The term “comprising” is not intended to limit inventions to only claiming the present invention with such comprising language. Any invention using the term comprising could be separated into one or more claims using “consisting” or “consisting of” claim language and is so intended.

Reference throughout this document to “one embodiment”, “certain embodiments”, and “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

The drawings featured in the figures are for the purpose of illustrating certain convenient embodiments of the present invention, and are not to be considered as limitation thereto. Term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

As used herein, the term “TATP residue” refers to any of the known forms of TATP, contaminates, masking agents and the like that will sublimate and are associated with the presence of TATP.

As used herein, the term “detection” refers to using the system and method of the present invention to determine if a TATP residue is present on a test subject. It includes a system for clearing the air and in one embodiment, for doing multiple successive testing of subjects.

As used herein, the term “enclosed sampling chamber” refers to an enclosure that is sufficiently large enough to hold the test subject or subjects and for the subject to enter and leave. In one embodiment, the chamber is about 3 m cubed or smaller for an average human subject. The size of the chamber can be adjusted based on the number of subjects and their size. Accordingly, one skilled in the art can vary the size based on how the air is concentrated, number of subjects and the like. In one embodiment, the sampling chamber is an airport X-ray machine or other airport security inspection chamber, i.e. making it a dual use machine. In another embodiment, the chamber is merely a room that is enclosed and of sufficient size as noted above. In one embodiment, it is of a size to enable, after concentration, one to achieve at least 1 part per million of TATP residue. In another embodiment, the enclosed sampling chamber is a virtual room with walls created by moving air, e.g. from an overhead drone, fans, or the like, which traps air in a particular area. The sample preparation system could be located anywhere, e.g. an airport, transportation hub, buildings, and the like. In one embodiment, it is in an open space with virtual walls created by moving air.

As used herein, the term “subject” refers to a living creature such as a human, pet, livestock, or the like or an inanimate object like luggage, and the like. The common element is that they are each being tested for TATP residue.

As used herein, the term “temperature regulator” refers to a device for holding or elevating the temperature in the sampling chamber to at least about 90 degree F. The temperature is selected to insure that TATP residue sublimates in the short period of time allowed for the test. Hotter air can be used when inanimate objects, that are not temperature sensitive, are tested. Heated air can be blowing in quickly to the chamber, though quick acting heating elements, or the like, are also contemplated. In one embodiment, jets of warmed air are directed at selected parts of the subject that might be more likely to have a TATP residue to increase the likelihood of detection. It is assumed, in one embodiment, there is a dwell time of the subject in the chamber of at least about 5 seconds to give sufficient time for the TATP to sublimate.

As used herein, the term “air sampler” refers to a device which removes an air sample from the sampling chamber after the dwell time. It is designed to deliver the sample to the device for concentrating the air sample. In all cases, these devices could be constructed together into one device that performs multiple tasks as outlined herein.

As used herein, the term “concentrating the air sample” refers to making sure that the air sample is concentrated enough that one would expect any TATP residue is at least 1 ppm (or to the limit of detection) based on enclosure sizes noted above. In one embodiment, the air sample is concentrated 5 times, 10 times, 15 times or more. One can concentrate the air samples in one or more ways and those ways are within the skill in the art, such as compression, refrigeration, e.g. around 32 to 36 degrees F., and small molecule gas phase separation of at least about 1:4. This system will also have a device for sending the concentrated sample to a TATP detector system.

As used herein, the term “system for concentrating the air” refers to taking the original sample and removing the original air or concentrating the sample, e.g. by putting the sample under pressure, refrigerating the sample or using small molecule gas phase separation. This and other methods are well within the skill in the art. The goal is to get the sample to at least 1 ppm or better or to the limit of detection of the detection system used in the present invention.

As used herein, the term “TATP detector system” refers to any one of the current or future systems which can detect a TATP residue in an air sample. Such systems are well known in the art. Those systems can, in addition, report the results to a user of the system sufficient to tell if a TATP residue is present or not with the limits of detection of the system. In one embodiment, it is an infrared detection system and in another embodiment, it is a mass spectrophotometry system.

### DRAWINGS

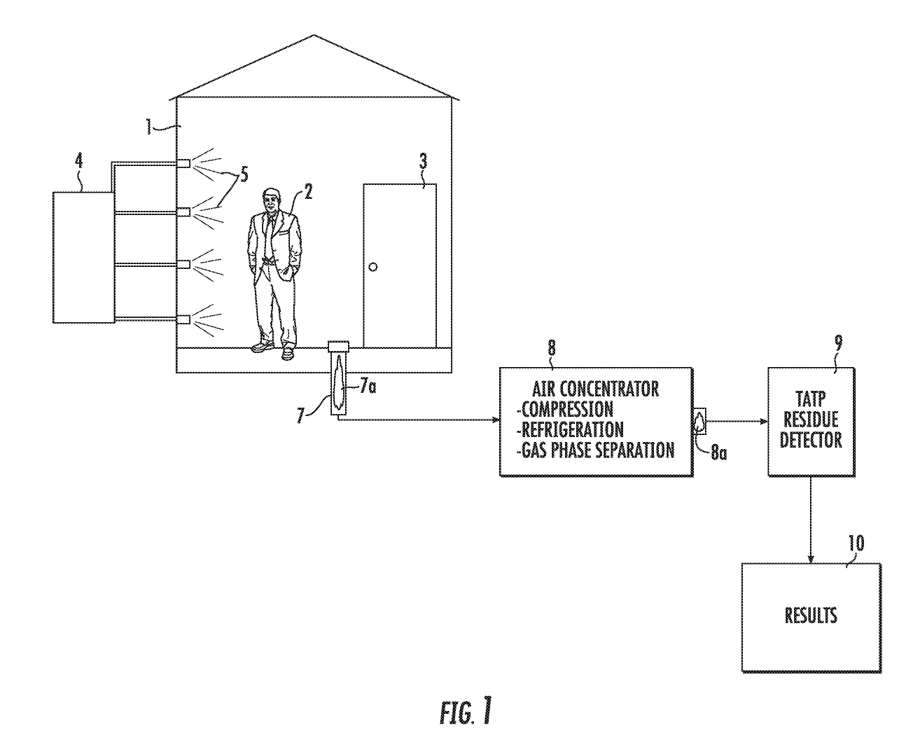

Now referring to the drawings, FIG. 1 is a diagram of the system of the present invention. In this view, an enclosed sampling chamber 1 contains a subject, in this view a human 2, who has entered through door 3. While this view shows a single subject 2, this is for convenience, and the use with multiple subjects and larger, smaller or virtual sampling chambers is intended. A temperature regulator 4 is shown which heats air quickly to at least about 90 degrees F. or more, and sprays jets of air 5 onto subject 2 to induce sublimation of any TATP residue on the subject. The subject remains in the chamber for a dwell time of at least about 2-5 seconds or more to allow TATP to sublimate and enter the heated atmosphere of the chamber 1. Air sampler 7 removes an air sample 7a after the appropriate dwell time. Air sampler 7 can also be used as an exhaust to remove resident air between testing of subjects. In one embodiment, an exhaust device can be separate from air sampler device. A portion of the air sample 7a is transferred to a concentrating device 8 which, in this embodiment, does each of, compression to about ten atmospheres, chilling to just above freezing (about 34 degree F.) and a gas phase separation. While this is shown as a single device, multiple devices can be utilized as desired. In addition, any other method of concentrating the air sample is within the scope of this invention. The concentrated sample 8a is then moved to a TATP residue detector 9 which samples the air sample 8a for the presence of TATP residue. In this example, the sample has been concentrated to at least about 1 ppm but, depending on the detector, other concentrations within the limits of detection can be utilized and are within the skill in the art in view of this disclosure. Lastly, the detector sends results 10 to a GUI screen, print out or the like indicating the presence or non-presence of a TATP residue.

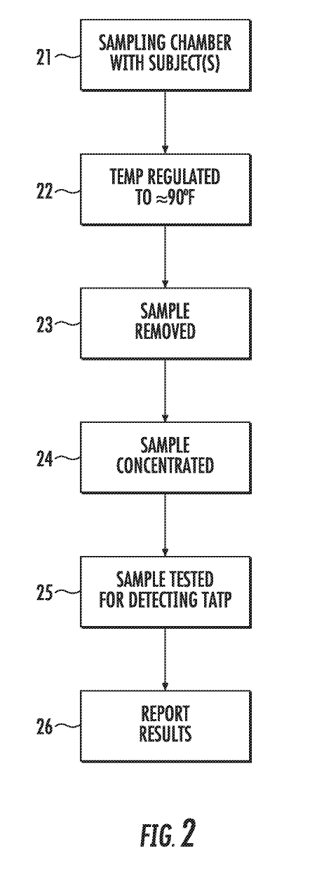

FIG. 2 shows a flow chart of the method of the present invention. In this Figure, a subject is placed in a sampling chamber 21, the chamber either has or is otherwise regulated to at least about 90 degrees F. 22. A sample of that air is taken 23 after a dwell time, the sample concentrated 24 and then sent to a TATP residue detector 25 for detection of TATP residue and then reports results 26.

Those skilled in the art to which the present invention pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present invention is, therefore, indicated by the appended claims rather than by the foregoing description or drawings. Consequently, while the present invention has been described with reference to particular embodiments, modifications of structure, sequence, materials and the like apparent to those skilled in the art still fall within the scope of the invention as claimed by the applicant.

What is claimed is:

1. A system for the detection of a triacetone-triperoxide residue on one or more subjects comprising:
a) an enclosed sampling chamber for positioning the subject;
b) a temperature regulator for providing a temperature of at least about 90 degrees F. in the sampling chamber;
c) an air sampler for taking air from the chamber after a dwell time of at least about 5 seconds; and
d) a system for concentrating the air sample and feeding it to a TATP detector system.

2. The system according to claim 1 which further comprises of a TATP detector system for receiving a concentrated air sample, determining if any TATP residue is present and reporting the results.

3. The system according to claim 1 wherein the method used to concentrate the air sample is at least one of air compression, refrigeration and gas phase separation.

4. The system according to claim 1 wherein the TATP detector system is a system which detects TATP residue by at least one of infrared or mass spectrophotometry methods.

5. The system according to claim 1 wherein the sampling chamber is an airport security inspection chamber.

6. The security system according to claim 1 wherein the subject is an object or a living creature.

7. The security system according to claim 1 wherein the sampling chamber is of a size large enough to accommodate a plurality of subjects.

8. The security system according to claim 1 which further comprises a device for clearing the air in the chamber to allow for successive testing of subjects.

9. The security system according to claim 1 wherein there are air jets to direct the air in the chamber toward the subject.

10. A method for the detection of a triacetone-triperoxide residue on one or more subjects comprising:
a) providing an enclosed sampling chamber for positioning the subject;
b) providing a temperature regulator for providing a temperature of at least about 90 degrees F. in the sampling chamber;
c) providing an air sampler which takes an air sample from the chamber after a dwell time of at least about 5 seconds; and
d) concentrating the air sample and feeding it to a TATP detector system.

11. The method according to claim 10 which further comprises of A TATP residue detector system for receiving a concentrated air sample, determining if any TATP residue is present and reporting the results.

12. The method according to claim 10 wherein the method used to concentrate the air sample is at least one of air compression, refrigeration and gas phase separation.

13. The method according to claim 10 wherein TATP detector system is a system which detects TATP residue by at least one of the methods of infrared or mass spectrophotometry methods.

14. The method according to claim 10 wherein the provided sampling chamber is an airport security inspection chamber.

15. The method according to claim 10 wherein the subject is an object or a living creature.

16. The method according to claim 10 wherein the sampling chamber is of a size large enough to accommodate a plurality of subjects.

17. The method according to claim 10 which further comprises a device for clearing the air in the chamber to allow for successive testing of subjects.

18. The method according to claim 10 which further comprises blowing jets of air within the chamber direct toward the subject.
